(19) United States
(12) Patent Application Publication
(10) Pub. No.: US 2013/0023931 A1
(43) Pub. Date: Jan. 24, 2013

(54) SPINAL TRACTION DEVICE

(71) Applicants: Nicholas John Bovell, Swanbourne (AU); Radu Iliuta, Saint-Jean (FR)

(72) Inventors: Nicholas John Bovell, Swanbourne (AU); Radu Iliuta, Saint-Jean (FR)

(21) Appl. No.: 13/639,336

(22) PCT Filed: Apr. 4, 2011
(86) PCT No.: PCT/AU11/00384

§ 371 Date: Oct. 4, 2012

(30) Foreign Application Priority Data
Apr. 7, 2010 (AU) 2010901469

(51) Int. Cl. A61F 5/042

(52) U.S. Cl. 606/241

(57) ABSTRACT

A spinal traction device (50) comprises a collar (52) adapted to fit snugly around a user's neck, the collar (52) comprising a pair of elongate collar members (56) which respectively pass around both side of the user's neck and provide a grip against the user's neck and posterior jaw. A suspension means (58) is provided for suspending the collar (52) from a secure anchor point above the user's head. In use, when the user wears the collar (52) around their neck, and allows at least part of their body weight to be suspended by the collar (52) from the anchor point, the device (50) can apply traction to the user's spine.

### FIELD OF THE INVENTION

The present invention relates to a spinal traction device for stretching a user's spine and neck muscles and relates particularly, though not exclusively, to such a device that relies on the force of gravity to provide the traction force.

### BACKGROUND TO THE INVENTION

Many people, healthy or injured, active or sedentary, at one point or another, experience discomfort or pain around the upper back, lower back and neck regions of the spine. Active individuals and sportspersons alike often experience discomfort or pain as a result of strain to the muscles or vertebra of the upper back, lower back and neck region. Many sportspersons require constant remedy for muscle aches and pains, and pains or problems resulting from compression or herniation of intervertebral discs. The neck, upper back and lower back are the most difficult regions to treat effectively. Stretching and spinal decompressions are some of the most effective measures to relieve tension and prevent future injury.

Many people who sit in front of a computer all day experience adverse effects such as a stiff neck and tight muscle's, commonly a result of poor sitting posture.

There are also many people who find that stretching the spine and associated muscles is a great relaxation exercise, similar in many respects to the therapeutic effects of yoga; the spine and neck being one of the few regions of the body that can only be stretched with the help of a device.

After a night's sleep people wake up 1-3 cm taller than when they went to sleep due to the decompressing effects of being prone. This proves vertical spinal height can be manipulated to some degree. Some people endeavour to increase their vertical height by stretching their spinal column. There is evidence to suggest this can be achieved, albeit the height increase is possibly only temporary.

Spinal decompression has been used extensively to treat all types of back pain for decades. People who have minor spinal injuries and back pain resulting from problems such as compressed vertebrae often find that spinal traction and decompression is an effective and relatively cost effective remedy.

Spinal traction decompresses the spinal column thereby increasing the space between each vertebra. This has a two-fold effect. Firstly, it immediately takes pressure off the spine and relieves pain that is caused by pressure to nerve roots. Secondly, if the decompression is maintained for a certain amount of time, say 20 min, the change in the pressure gradient surrounding each vertebral disc allows the disc to take in surrounding interstitial fluid and swell larger, and take in vital nutrients which enhance healing. This disc augmentation lasts long after the decompression is stopped and this prolongs any increased comfort associated with the relief of pressure.

Like any part of the human body, the spine must be regularly moved, stretched and put into traction in order for it to stay supple, relieve tension and prevent injury.

A number of prior art traction devices are available for applying traction to the spine and/or neck. These prior art devices typically involve lying on a table, bench or other piece of equipment which is designed to stretch the spine by inversion. A disadvantage of inversion devices is the danger of excess blood pressure in the veins of the head, leading to discomfort and possible burst veins. Other devices employ a harness that is attached to the skull or chest of the user and various sized weights are applied to provide the traction force. A disadvantage of most prior art devices is that they do not act to separate all the vertebrae of the spine, from the neck to the tailbone. Other spinal decompression remedies require visiting a specialist and being put onto a traction machine which is extremely costly and inconvenient.

The present invention was developed with a view to providing a spinal traction device that provides more comprehensive decompression of the spine than prior art devices, and that is simple to use.

References to prior art in this specification are provided for illustrative purposes only and are not to be taken as an admission that such prior art is part of the common general knowledge in Australia or elsewhere.

### SUMMARY OF THE INVENTION

According to one aspect of the present invention there is provided a spinal traction device for stretching a user's spine, the device comprising:

a collar adapted to fit snugly around a user's neck, the collar having a cushioning means where it makes contact with the user's jaw and neck;

suspension means for suspending the collar from a secure anchor point above the user's head whereby, in use, when the user wears the collar around their neck, and allows at least part of their body weight to be suspended by the collar from the anchor point, the device can apply traction to the user's spine.

Preferably the device further comprises a pair of underarm support means which is connected to the collar and is adapted to extend underneath the armpits of the user and against their upper rib cage. The underarm support means is preferably padded and is adapted to provide additional support for the user.

Preferably the pair of underarm support means comprises a pair of elongate support frame members pivotally coupled to each other at a pivot point to form a scissor frame. Typically one end of each support frame member has an underarm support which is contoured to fit the user's body beneath the armpits and against their upper rib cage. Preferably the other end of each support frame member is coupled to the suspension means. Advantageously when the device is suspended from the anchor point the scissor frame causes the underarm supports on each support frame member to press downwards against the upper rib cage.

Preferably the collar comprises a pair of elongate collar members which respectively pass around both side of the user's neck and provide a grip against the user's neck and posterior jaw. Preferably the collar is also provided with adjusting means for adjusting the size of the collar. Preferably a front end of each collar member is coupled to the other by an adjustable tension strap which locates adjacent to the user's chin. Loosening and tightening, of the tension strap adjusts the grip of the collar against the user's neck and jaw.

Preferably a back end of each collar member is mechanically coupled to the scissor frame. Preferably the back ends of the collar members are joined together and coupled to the pivot point of the scissor frame. Advantageously the pivot point comprises a pivot hub on which both support frame members are pivotally received. Preferably the pivot hub has a collar support member diametrically mounted therein to which the collar is slidably coupled. Advantageously the vertical position of the collar on the collar support member is adjustable.

In one embodiment of the invention, when the user wears the collar around their neck, and the user allows substantially their full body weight to be suspended by the collar from the anchor point, the device can apply traction to the user's spine.

In an alternative embodiment the collar comprises a means for opening the collar at the front, allowing the user to place it around their neck, the collar having fastening means for securely closing the opening means once the collar is fitted around the user's neck. Typically the fastening means comprises a plurality of fastening clips or clasps.

In this embodiment the collar comprises an external collar shell, preferably made from a moulded plastics material. Advantageously the collar shell is moulded with a narrowed neck portion, and is flared upwards to form an enlarged upper opening in which the user's chin and jaw can be supported. Preferably the upper opening is arranged at an inclined angle, with the rear of the opening, where it contacts a base of the skull, higher than the front where it contacts the user's chin.

Typically the suspension means comprises a main first strap, which is secured to the device by two converging straps.

Throughout the specification, unless the context requires otherwise, the word “comprise” or variations such as “comprises” or “comprising”, will be understood to imply the inclusion of a stated integer or group of integers but not the exclusion of any other integer or group of integers. Likewise the word “preferably” or variations such as “preferred”, will be understood to imply that a stated integer or group of integers is desirable but not essential to the working of the invention.

### BRIEF DESCRIPTION OF THE DRAWINGS

The nature of the invention will be better understood from the following detailed description of several specific embodiments of the spinal traction device, given by way of example only, with reference to the accompanying drawings, in which:

FIG. 1 is a side elevation of a first embodiment of the spinal traction device according to the invention;

FIG. 2 is a front elevation of the spinal traction device of FIG. 1;

FIG. 3 is a side elevation illustrating the manner in which the spinal traction device of FIG. 1 is worn by a user;

FIG. 4 is a rear elevation illustrating the manner in which the spinal traction device of FIG. 1 is worn by a user;

FIG. 5 illustrates one possible method of using the spinal traction device of FIG. 1;

FIG. 6 is a front top perspective view of a second embodiment of the spinal traction device according to the invention;

FIG. 7 is another front perspective view of the spinal traction device of FIG. 6;

FIG. 8 is a back top perspective view of the spinal traction device of FIG. 6; and,

FIG. 9 illustrates the spinal traction device of FIG. 6 worn by a user.

### DETAILED DESCRIPTION OF PREFERRED EMBODIMENTS

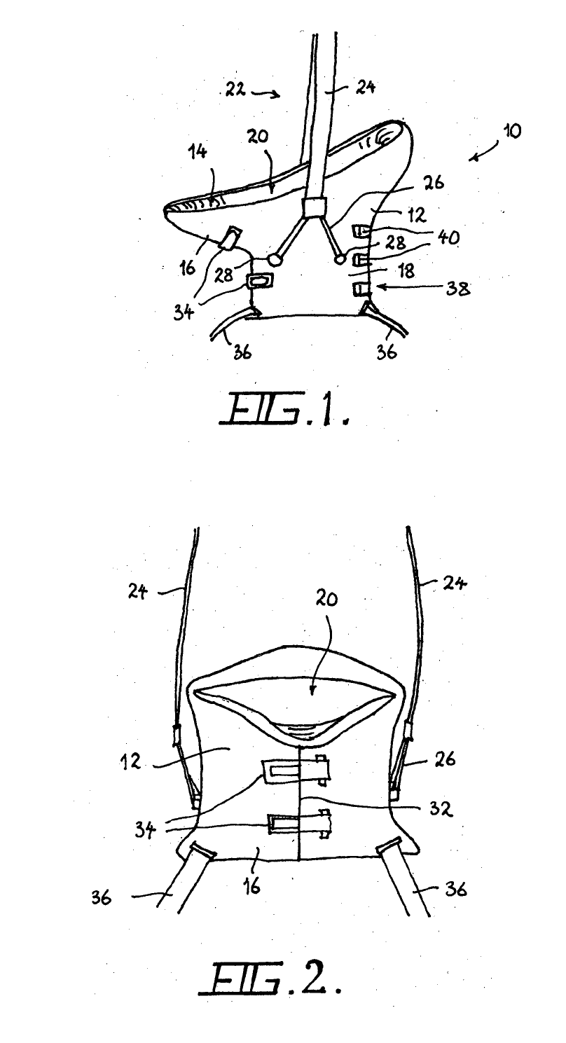

A first embodiment of spinal traction device 10 in accordance with the invention, as illustrated in FIGS. 1 to 5, comprises a collar 12 adapted to fit snugly around a user's neck. The collar 12 has an interior cushioning means 14 where it makes contact with the user's jaw, chin and neck. Preferably the cushioning means 14 comprises a layer of foamed cushioning material made from, for example, foamed plastics material or latex. Preferably the foamed cushioning material has a relatively high stiffness to provide adequate support for the user's jaw, chin and neck.

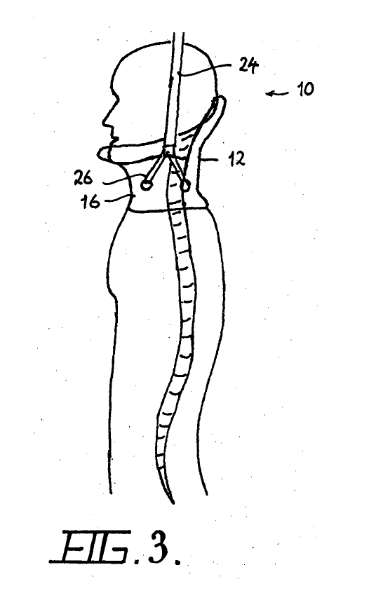

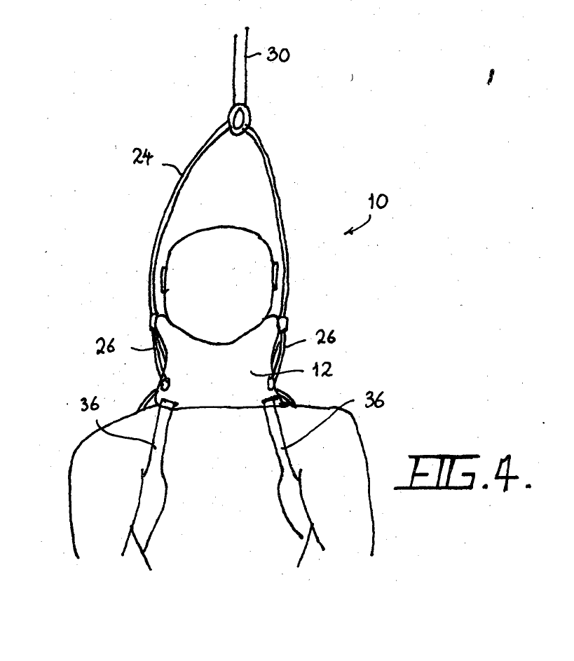

Preferably the collar 12 further comprises an external collar shell 16, preferably made from a moulded plastics material, for example, a thermoplastic material of the kind used for making splints. As can be seen most clearly in FIGS. 1 and 2, the collar shell is moulded with a narrowed neck portion 18, and then is flared upwards to form an enlarged upper opening 20 in which the user's chin and jaw can also be supported. Typically the upper opening 20 is flared in one plane at both the front and back of the collar 12, and is adapted to allow the collar 12 to also contact the base of the skull at the rear of the user's head. For this purpose the upper opening 20 is preferably arranged at an inclined angle, with the rear of the opening 20, where it contacts the base of the skull, higher than the front where it contacts the user's chin (see also FIG. 3). The angle of inclination of the upper opening 20 is designed to ensure the user's head is supported at the correct angle during traction.

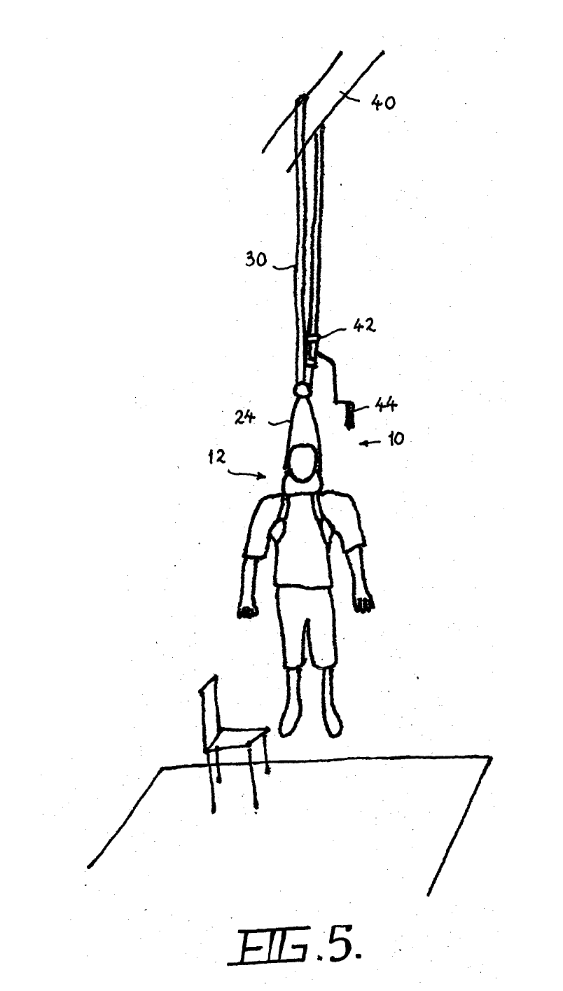

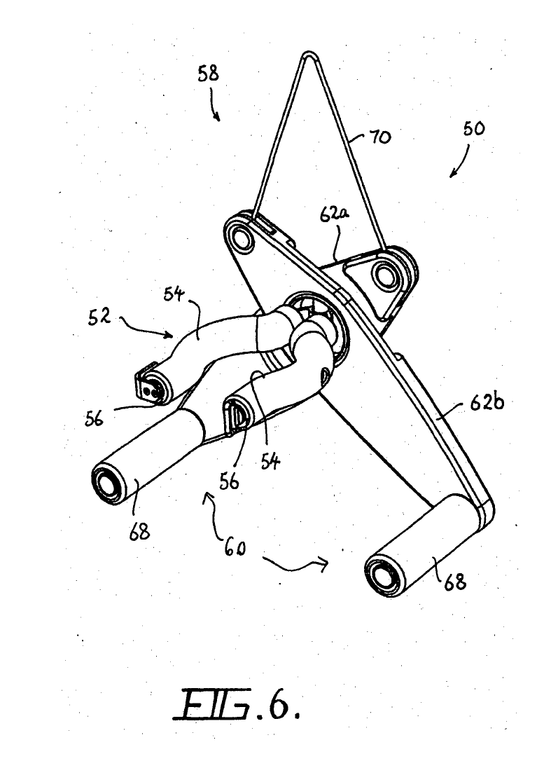

The spinal traction device 10 further comprises a suspension means 22 for suspending the collar 12 from a secure anchor point above the user's head. In use, when the user wears the collar 12 around their neck, and allows at least part of their body weight to be suspended by the collar from the anchor point, the device can apply traction to the user's spine. In this embodiment, when the user wears the collar 12 around their neck, and allows substantially their full body weight to be suspended by the collar from the anchor point, the device 10 can apply traction to the user's spine (see FIG. 5). Typically the suspension means 22 comprises a main first strap 24, which is secured to the collar shell 16 by two converging straps 26 which are coupled to the respective ends of the main first strap 24 (see FIG. 1). The respective ends of the converging straps 26 are preferably fastened to the collar shell 16 by suitable bolts 28. The main strap 24 is typically attached to another rope or cable 30 for connection to an anchor point (see FIGS. 4 and 5).

Preferably the collar 12 further comprises an opening means 32 for allowing the user to place it around their neck. In the illustrated embodiment the opening means comprises a cut 32 provided in the front of the collar shell 16 which allows the front of the collar 12 to be opened to form a gap through which the user's neck may be received. The collar shell 16 is provided with fastening means 34 for securely closing the front of the collar 12 once it is fitted around the user's neck. Typically the fastening means comprises a plurality of fastening clips or clasps 34, which may be similar in design to that used on ski-boots.

Preferably the device 10 further comprises a pair of underarm support means which is connected to the collar and is adapted to extend underneath the armpits of the user and against their upper rib cage. In this embodiment the underarm support means comprises a pair of underarm straps 36 which are connected to the lower edge of the collar 12 and are adapted to loop underneath the armpits of the user and against their upper rib cage, as shown in FIGS. 4 and 5. The underarm straps 36 are preferably padded and are adapted to provide additional support for the user in the event that they cannot handle the strain on their neck of their full body weight.

Preferably the collar 12 is also provided with adjusting means 38 for adjusting the size of the collar. The adjusting means 38 preferably comprises a plurality of size adjustment screws 40 which allow the user to adjust the size of the collar shell 16 prior to fitting around their neck. The adjusting screws 40 allow the internal circumference, and hence the diameter, of the collar 12 to be adjusted to suit different people's necks.

A typical method of using the spinal traction device 10 will now be described with reference to FIGS. 4 and 5. The user typically first fits the collar 12 to their neck and makes any size adjustments needed to ensure it fits snugly and provides full support for the neck, jaw and chin. The collar 12 is then secured by closing the fastening clasps 34. If needed, the underarm straps 36 may be placed under the arms and adjusted to provide additional support.

The cable or rope 30 is fixed to an anchor point (roof rafter 40 in FIG. 5). The length of the rope 30 is selected so that when the main strap 24 is attached to it, the collar 12 will be suspended at a height where the user's feet cannot touch the ground. The use then steps onto a stool or chair and hitches the main strap 24 onto the rope 30. The user then carefully steps off the stool or chair, gently allowing his or her full body weight to gradually take up the tension on the strap (see FIG. 5).

Alternatively, the rope 30 may be fitted with a simple ratchet device 42 with a reachable handle 44 (see also FIG. 5). Instead of stepping off a stool or chair, the user turns the ratchet handle 44 to gradually increase the tension applied to the strap 24. This causes the user to be gradually lifted-up off the ground until they are completely suspended with their full body applying traction to their spine. The use of the ratchet device 42 has the added advantage that the amount of traction applied to the spine can be adjusted to less than the full body weight, by the user keeping his or her feet touching the ground and bearing some of their body weight through their legs.

The spinal traction device can be attached to a rope or cable which is looped over a variety of overhead anchor points, including rafters, chin-up bars, boxing bag fixtures, or even sturdy tree branches, provided the anchor point is sufficiently secure to support the full body weight of the person being lifted.

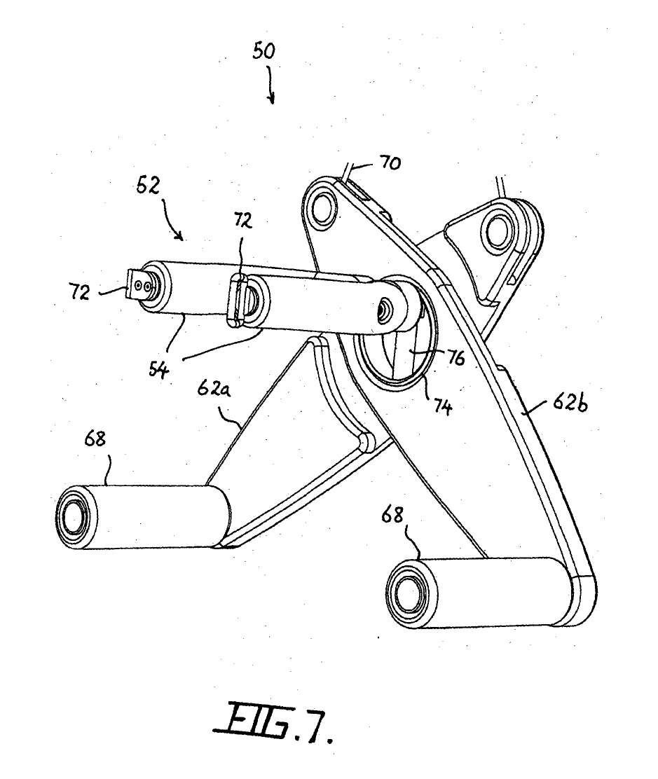

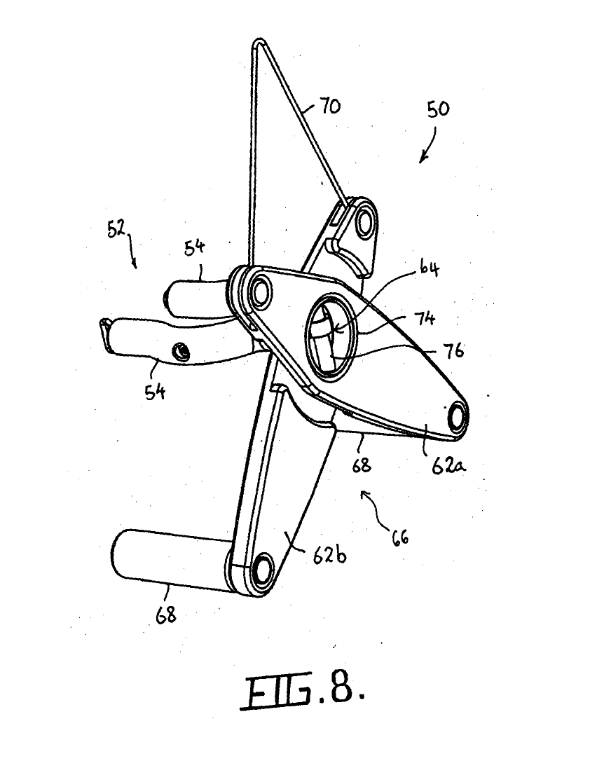

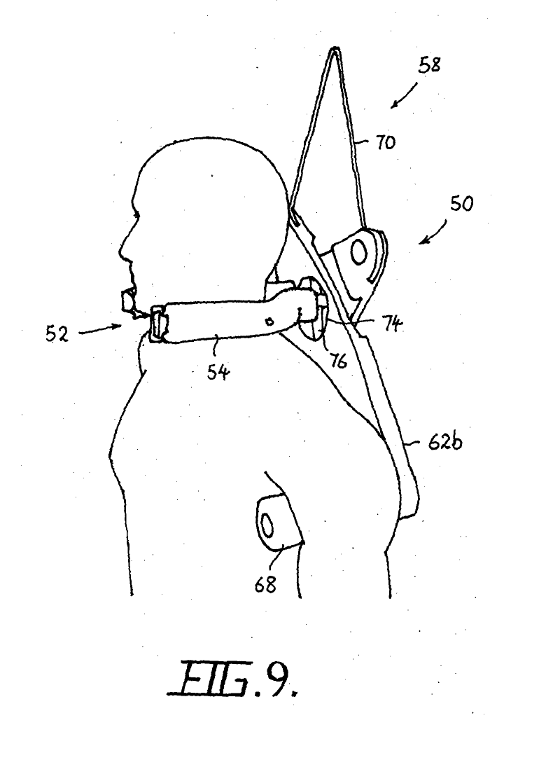

A second embodiment of the spinal traction device 50 will now be described with reference to FIGS. 6 to 9. The spinal traction device 50 comprises a collar 52 adapted to fit snugly around a user's neck, the collar having an cushioning means 54 where it makes contact with the user's jaw and neck. In this embodiment the collar 52 comprises a pair of elongate collar members 56 which respectively pass around both side of the user's neck and provide a grip against the user's neck and posterior jaw (see FIG. 9). Each collar member 56 is made from 25 mm steel pipe, 2 mm thick and is covered by a foam core which is then covered by soft cotton to provide the cushioning means 54 where it contacts the user's skin.

The device further comprises' a suspension means 58 for suspending the collar 52 from a secure anchor point above the user's head. In use, when the user wears the collar 52 around their neck, and allows at least part of their body weight to be suspended by the collar 52 from the anchor point, the device 50 can apply traction to the user's spine.

Preferably the device further comprises a pair of underarm support means 60 which is connected to the collar 52 and is adapted to extend underneath the armpits of the user and against their upper rib cage. The underarm support means 60 is preferably padded and is adapted to provide additional support for the user. In this embodiment the pair of underarm support means 60 comprises a pair of elongate support frame members 62 (shown as 62a and 62b) pivotally coupled to each other at a pivot point 64 to form a scissor frame 66.

One end of each support frame member 62 has fixed thereto an underarm support 68 which is contoured to fit the user's body beneath the armpits and against their upper rib cage. Typically each underarm support 68 is covered by a foam core and soft cotton to provide cushioning. Preferably the other end of each support frame member 62 is coupled to the suspension means 58. Suspension means 58 of this embodiment comprises a cable 70 which is attached at its respective ends to the other end of each support frame member 62. Hence when tension is applied to the cable 70, it will not only apply an upwards force to the support frame members 62 of the scissor frame 66, but also an inward force as the two support frame members 62 pivot about the pivot point 64.

Hence when the device 50 is suspended from an anchor point via the cable 70 the scissor frame 66 causes the underarm supports 68 on each support frame member 62 to press downwards against the upper rib cage, thus eliminating some of the discomfort of being supported under the arms by a solely vertical force. Also, because the collar 52 is attached to the scissor frame 66 it causes the neck to be pushed away from the body as the tension on the cable 70 causes the support frame members 62 to close iri. The vertical spacing between the collar 52 and the underarm supports 68 may thus increase from anywhere between 10% and 20% so as to apply additional traction to the neck relative to the body.

Preferably the collar 52 is also provided with adjusting means for adjusting the size of the collar. A front end of each collar member 56 is coupled to the other by an adjustable tension strap (not shown) which locates adjacent to the user's chin. Suitable fasteners 72 are provided at the front end of the respective collar members 56 for holding the tension strap. Loosening and tightening of the tension strap adjusts the grip of the collar 52 against the user's neck and jaw. When the strap is tightened it causes the collar members 56 to flex inwards providing a snug grip around the user's neck and posterior jaw. The ability for adjustments at the back and front of the collar 52 accommodates people with different neck diameters.

The back end of each collar member 56 is mechanically coupled to the scissor frame 66. In this embodiment the back ends of the respective collar members 56 are joined together and coupled to the pivot point 64 of the scissor frame 66. Advantageously the pivot point comprises a pivot hub 74 on which both support frame members 62 are pivotally received. Each support frame member 62 has a circular aperture provided intermediate its length, which is received on the outer circumference of the pivot hub 74. Preferably the pivot hub 74 is hollow and has a collar support member 76 diametrically mounted therein to which the collar 52 is slidably coupled.

Advantageously the vertical position of the collar 52 on the collar support member 76 is adjustable. Vertical adjustment of the collar 52 where it sits in relation to the scissor frame 66 allows people of different neck lengths to use the device appropriately. It is possible that the adjustments can be done with the use of a remote control device thus allowing the user to find the correct adjustment while wearing the spinal traction device 50. Other options for adjustment are a spring or fluid actuator activated by a hand pump, or simply a manual setting.

Once the suspension means 58 is secured onto an anchor the user can enter the spinal traction device 50 at a height where their heels are off the ground, standing on the balls of their feet. After making all necessary adjustments the user can lower their heels allowing tension to take up on the device 50. This will put the user's spine into traction and facilitate decompression, also stretching associated neck and back muscles. The degree to which the user wishes to be suspended is entirely up to them. A full suspension can be achieved. The spinal traction device 50 is comfortable enough to be used for extended periods of time, for example, from five minutes to an hour.

It is possible the device 50 will include a tension limiter in the collar adjustment to prevent excess tension being put on the neck. This is a safety precaution. Another safety feature could be an emergency release knob.

Now that several embodiments of the spinal traction device have been described in detail, it will be apparent that the described embodiments provide a number of advantages over the prior art, including the following:
(i) It allows each and every vertebral disc to separate from one another, from the cervical vertebrae (neck) to the coccygeal vertebrae (tailbone), thereby providing a more comprehensive stretch than any other method.
(ii) By keeping the user in an upright position there is none of the discomfort and potential problems caused by inversion devices.
(iii) It provides more effective relief from pain and discomfort by providing decompression of vertebrae and stretching of the back and neck muscles.
(iv) It can be used simply and safely and for personal use within the home; it's not big or cumbersome; no professional supervision is needed; and, it's good for sports therapy.

It will be readily apparent to persons skilled in the relevant arts that various modifications and improvements may be made to the foregoing embodiments, in addition to those already described, without departing from the basic inventive concepts of the present invention. For example, the design of the collar may vary significantly from that shown. Therefore, it will be appreciated that the scope of the invention is not limited to the specific embodiments described.

1. A spinal traction device for stretching a user's spine, the device comprising:
a collar adapted to fit snugly around a user's neck, the collar having a cushioning means where it makes contact with the user's jaw and neck;
suspension means for suspending the collar from a secure anchor point above the user's head whereby, in use, when the user wears the collar around their neck, and allows at least part of their body weight to be suspended by the collar from the anchor point, the device can apply traction to the user's spine.

2. A spinal traction device as defined in claim 1, further comprising a pair of underarm supports that are connected to the collar and are adapted to extend underneath the armpits of the user and against their upper rib cage.

3. A spinal traction device as defined in claim 2, wherein the underarm supports are padded and are adapted to provide additional support for the user.

4. A spinal traction device as defined in claim 2, wherein the pair of underarm supports comprises a pair of elongate support frame members pivotally coupled to each other at a pivot point to form a scissor frame.

5. A spinal traction device as defined in claim 4, wherein one end of each support frame member has an underarm support which is contoured to fit the user's body beneath the armpits and against their upper rib cage.

6. A spinal traction device as defined in claim 5, wherein the other end of each support frame member is coupled to the suspension means.

7. A spinal traction device as defined in claim 6, wherein when the device is suspended from the anchor point the scissor frame causes the underarm supports on each support frame member to press downwards against the upper rib cage.

8. A spinal traction device as defined in claim 4, wherein the collar comprises a pair of elongate collar members which respectively pass around both side of the user's neck and provide a grip against the user's neck and posterior jaw.

9. A spinal traction device as defined in claim 8, wherein the collar is also provided with adjusting means for adjusting the size of the collar.

10. A spinal traction device as defined in claim 9, wherein a front end of each collar member is coupled to the other by an adjustable tension strap which locates adjacent to the user's chin.

11. A spinal traction device as defined in claim 10, wherein loosening and tightening of the tension strap adjusts the grip of the collar against the user's neck and jaw.

12. A spinal traction device as defined in claim 8, wherein a back end of each collar member is mechanically coupled to the scissor frame.

13. A spinal traction device as defined in claim 12, wherein the back ends of the collar members are joined together and coupled to the pivot point of the scissor frame.

14. A spinal traction device as defined in claim 13, wherein the pivot point comprises a pivot hub on which both support frame members are pivotally received.

15. A spinal traction device as defined in claim 14, wherein the pivot hub has a collar support member diametrically mounted therein to which the collar is slidably coupled.

16. A spinal traction device as defined in claim 15, wherein the vertical position of the collar on the collar support member is adjustable.

17. A spinal traction device as defined in claim 1, wherein the collar comprises a means for opening the collar at the front, allowing the user to place it around their neck, the collar having fastening means for securely closing the opening means once the collar is fitted around the user's neck.

18. A spinal traction device as defined in claim 17, wherein the fastening means comprises a plurality of fastening clips or clasps.

19. A spinal traction device as defined in claim 17, wherein the collar comprises an external collar shell, preferably made from a moulded plastics material.

20. A spinal traction device as defined in claim 19, wherein the collar shell is moulded with a narrowed neck portion, and is flared upwards to form an enlarged upper opening in which the user's chin and jaw can be supported; optionally wherein the upper opening is arranged at an inclined angle, with the rear of the opening, where it contacts a base of the skull, higher than the front where it contacts the user's chin.

21.-22. (canceled)
